SEQUENCE LISTING (11 sequences)

SEQ ID NO: 1 - DNA, 24 bp, Artificial Sequence
  Primer
gcatggacatcgacccttataaag

SEQ ID NO: 2 - DNA, 25 bp, Artificial Sequence
  Primer
ggagctactgtggagttactcctgg

SEQ ID NO: 3 - DNA, 20 bp, Artificial Sequence
  Primer
gttcaagcctccaagctgtg

SEQ ID NO: 4 - DNA, 25 bp, Artificial Sequence
  Primer
tcagaaggcaaaaaagagagtaact

SEQ ID NO: 5 - DNA, 46 bp, Artificial Sequence
  Primer
tttttttttttttttcgtgcgttcaagcctccaagctgtggcacga

SEQ ID NO: 6 - DNA, 20 bp, Artificial Sequence
  Primer
gttcaagcctccaagctgtg

SEQ ID NO: 7 - DNA, 25 bp, Artificial Sequence
  Primer
tcagaaggcaaaaaagagagtaact

SEQ ID NO: 8 - DNA, 24 bp, Artificial Sequence
  Primer
gcatggacatcgacccttataaag

SEQ ID NO: 9 - DNA, 18 bp, Artificial Sequence
  Primer
tctttataagggtcgcct

SEQ ID NO: 10 - DNA, 20 bp, Artificial Sequence
  Primer
gttcaagcctccaagctgtg

SEQ ID NO: 11 - DNA, 25 bp, Artificial Sequence
  Primer
tcagaaggcaaaaaagagagtaact

(19) United States
(12) Patent Application Publication
(10) Pub. No.: US 2017/0354967 A1
(43) Pub. Date: Dec. 14, 2017

(54) LAB-ON-CHIP SYSTEM FOR ANALYZING NUCLEIC ACID

(71) Applicants: CapitalBio Corporation, Beijing (CN); Tsinghua University, Beijing (CN)

(72) Inventors: Shengce TAO, Beijing (CN); Jing CHENG, Beijing (CN); Xuemei MA, Beijing (CN); Qiong ZHANG, Beijing (CN); Yuxiang ZHOU, Beijing (CN)

(73) Assignees: CapitalBio Corporation, Beijing (CN); Tsinghua University, Beijing (CN)

(21) Appl. No.: 15/679,111

(22) Filed: Aug. 16, 2017

Related U.S. Application Data
(62) Division of application No. 10/547,742, filed on Apr. 7, 2006.

(30) Foreign Application Priority Data
Mar. 3, 2003 (CN) 03105108.1

(51) Int. Cl. B01L 3/00; C12Q 1/68; C40B 40/06; B01L 7/00; C40B 60/14

(52) U.S. Cl. CPC B01L 3/508; C12Q 1/6837; B01J 2219/00385; C40B 60/14; B01J 2219/0061; B01J 2219/00722; B01L 2300/0822; B01J 2219/00626; B01J 2219/00527; B01L 7/52; B01L 2300/0636; C40B 40/06; B01J 2219/00608; B01J 2219/00612; B01J 2219/0063; B01L 2300/163

(57) ABSTRACT

This invention relates generally to the field of nucleic acid detection. In particular, the invention provides a lab-on-chip system for analyzing a nucleic acid, which system comprises, inter alia, controllably closed space, and a target nucleic acid can be prepared and/or amplified, and hybridized to a nucleic acid probe, and the hybridization signal can be acquired if desirable, in the controllably closed space without any material exchange between the controllably closed space and the outside environment. Methods for analyzing a nucleic acid using the lab-on-chip system is also provided.

### CROSS-REFERENCE TO RELATED APPLICATIONS

This application is a divisional application of U.S. patent application Ser. No. 10/547,742, filed Sep. 1, 2005, which is a national phase of PCT application PCT/CN2003/000328 having an international filing date of May 6, 2003, which claims priority from Chinese patent application number 03105108.1 filed Mar. 3, 2003. The contents of these documents are incorporated herein by reference in their entireties.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 514572000910SeqList.txt, date recorded: Aug. 16, 2017, size: 2,262 bytes).

### TECHNICAL FIELD

This invention relates generally to the field of nucleic acid detection. In particular, the invention provides a lab-on-chip system for analyzing a nucleic acid, which system comprises, inter alia, controllably closed space, and a target nucleic acid can be prepared and/or amplified, and hybridized to a nucleic acid probe, and the hybridization signal can be acquired if desirable, in the controllably closed space without any material exchange between the controllably closed space and the outside environment. Methods for analyzing a nucleic acid using the lab-on-chip system is also provided.

### BACKGROUND ART

In the current methods for detecting infectious agents, cell culturing is mainly used for detecting infectious bacteria and serology is mainly used for detecting infectious virus. Nucleic acid based detection methods are rapid, sensitive and may shorten or even eliminate waiting period comparing to the traditional detection methods, e.g., cell culturing or serology based methods. Therefore, nucleic acid based detection methods are natural trends for clinical detections.

Traditional nucleic acid based detection methods, especially clinical detection methods for infectious agents, include three separate steps. The first step is sample preparation, e.g., treating samples, such as serum, whole blood, saliva, urine and faeces, to obtain nucleic acids, e.g., DNA or RNA. Often, insufficient amount of the nucleic acids are isolated or prepared from the samples and the prepared nucleic acids are amplified using a number of methods such as polymerase chain reaction (PCR), reverse transcription polymerase chain reaction (RT-PCR), strand displacement amplification

(SDA) and rolling cycle amplification (RCA), etc. (Andras et al., Mol. Biotechnol., 19:29-44 (2001)). The second step is hybridization as the conventional electrophoresis analysis is not sufficiently specific and hybridization is normally required for clinical detection methods. Exemplary hybridization methods include Northern blot, dot blot (or dot hybridization) and slot blot (or slot hybridization). The third step is to detect the hybridization signal, which is often based on the detection of a label. The label can be introduced during the amplification or hybridization step. The signal detection methods vary according to the label used, e.g., a fluorescent detector is used to detect a fluorescent label, autoradiography is used to detect a radioactive label, and detection of a biolabel, e.g., biotin label, digoxigenin label, etc., may require further enzymatic amplifications.

Depending on the required detection sensitivity, various signal amplification methods can be used, e.g., Tyramide signal amplification (TSA) (Karsten et. al., Nucleic Acids Res., E4. ((2002)) and Dendrimer (Kricka Clin. Chem., 45:453-8 (1999)).

The separation of the three key steps in nucleic acid detection requires manual manipulations among these steps. Theses manual manipulations make the detection procedure complex, time consuming, costly, and may introduce experimental error, and decrease repeatability and consistency of the detection. The manual manipulations also increase cross contamination, which is a major reason that hampers wide application of nucleic acid based detection, especially any such detection comprising an amplification step, in clinical use.

Nucleic acid chip or array can be used to assay large number of nucleic acids simultaneously (Debouck and Goodfellow, Nature Genetics, 21 (Suppl.):48-50 (1999); Duggan et al., Nature Genetics, 21 (Suppl.):10-14 (1999); Gerhold et al., Trends Biochem. Sci., 24:168-173 (1999); and Alizadeh et al., Nature, 403:503-511 (2000)). Gene expression pattern under a given condition can be rapidly analyzed using nucleic acid chip or array. The SNPs in a particular region, up to a 1 kb, can be analyzed in one experiment using nucleic acid chip or array (Guo et al., Genome Res., 12:447-57 (2002)).

Biochemical reactions and analyses often include three steps: sample preparation, biochemical reactions and signal detection and data analyses. Miniaturizing one or more steps on a chip leads to a specialized biochip, e.g., cell filtration chip and dielectrophoresis chip for sample preparation, DNA microarray for detecting genetic mutations and gene expression and high-throughput micro-reaction chip for drug screening, etc. Efforts have been made to perform all steps of biochemical analysis on chips to produce micro-analysis systems or lab-on-chip systems. Using such micro-analysis systems or lab-on-chip systems, it will be possible to complete all analytic steps from sample preparation to obtaining analytical results in a closed system rapidly. One drawback of the current lab-on-chip systems is its requirement of complex micro-scale engineering, which is technologically demanding. Most of the reported lab-on-chip systems are based on the miniaturization of a particular step, e.g., sample preparation chip, (Wilding et al., Anal. Biochem., 257:95-100 (1998)), cell isolation chip (Wang et al., J. Phys. D: Appl. Phys., 26:1278-1285 (1993)) and PCR chip (Cheng et al., Nucleic Acids Res., 24:380-385 (1996)). Cheng et al. reported a first lab-on-chip system that integrates the sample preparation, biochemical reaction and result detection together (Cheng et al., Nat. Biotechnol., 16:541-546 (1998)), which has not been commercialized.

The currently commercialized system, e.g., Nanogen's Microelectronic Array, only integrates and automates the hybridization and signal detection steps. A set of complex instruments and analytical softwares must be used with the Nanogen's Microelectronic Array. In addition, the cost for making and using Nanogen's electrophoresis chip is high. The present application address the drawbacks of the existing lab-on-chip systems and other related issues in the art by providing a novel lab-on-chip system.

### DISCLOSURE OF THE INVENTION

In one aspect, the present invention is directed to a lab-on-chip system for analyzing a nucleic acid, which system comprises a controllably closed space enclosed by a suitable material on a substrate, wherein said suitable material is thermoconductive, biocompatible and does not inhibit nucleic acid amplification or hybridization, and said controllably closed space comprising, on the surface of said substrate, a nucleic acid probe complementary to a target nucleic acid and, on or off the surface of said substrate, other reagents suitable for preparation of said target nucleic acid from a sample, amplification of said target nucleic acid, hybridization between said nucleic acid probe and said target nucleic acid, and/or means for detecting hybridization between said nucleic acid probe and said target nucleic acid, and wherein addition of a sample comprising said target nucleic acid into said controllably closed space, under suitable conditions, results in continuous sample preparation from said sample and/or amplification of said prepared target nucleic acid, and hybridization between said nucleic acid probe and said target nucleic acid, and preferably the detection of the hybridization signal, in said controllably closed space without any material exchange between said controllably closed space and the outside environment.

In another aspect, the present invention is directed to a method for analyzing a nucleic acid, which method comprises: a) providing an above-described lab-on-chip system; b) adding a sample containing or suspected of containing a target nucleic acid into said controllably closed space of said system provided in a); and c) allowing continuous sample preparation of said target nucleic acid from said sample and/or amplification of said prepared target nucleic acid, and hybridization between said nucleic acid probe and said prepared target nucleic acid, and preferably the detection of the hybridization signal, in said controllably closed space.

### BRIEF DESCRIPTION OF THE DRAWINGS

FIG. 1 is a schematic diagram of an exemplary lab-on-chip system. The system includes: a nucleic acid amplification and hybridization chamber 1, a nucleic acid amplification and hybridization system 2, a probe 3 immobilized on a substrate, a solid substrate 4, a temperature control device 5 for controlling temperature of PCR reaction and hybridization, and a fluorescence scanner 6 for detection of hybridization signal. The amplification and hybridization chamber 1 is made of air-tight material which can stand temperature over 95° C. for a long time. The material is also thermoconductive and biocompatible, and does not inhibit nucleic acid amplification or hybridization. One example is MJ Research self seal chamber, a self seal gel, and an enclosed plastic lumen. The nucleic acid amplification and hybridization system 2, which allows proper nucleic acid amplification and hybridization, includes primers, a sample to be tested, and an optimized buffer system. The nucleic acid probe 3 can be immobilized on a chemically modified surface of a chip via covalent bond for specific detection of a complementary interaction with a target sequence. The substrate 4 is a thermoconductive, with a good strength, and biocompatible after chemical modification. The substrate does not inhibit nucleic acid amplification or hybridization. The material for the substrate is preferably easily obtainable and inexpensive. Suitable solid material includes glass, quartz glass, silicon, ceramic, plastic, and etc. The temperature controlling device 5 can control the rate of temperature increase and decrease and precision of temperature control. The device can be a commercially available PCR machine, an in situ PCR machine, or a micro-temperature control device for miniaturization of the whole system. The fluorescent scanner 6 can be a commercially available fluorescent scanner or a fluorescent micro-scanner.

FIG. 2A illustrates a state before a probe, for use in an integrated hybridization and detection system, is hybridized to a target molecule. The probe has a stem-loop structure. Because of the close proximity of the fluorophore at one end of the stem and a fluorescent quencher at the other end of the stem, the fluorescence emission from the fluorophore excited by a light source is quenched by the quencher and no signal can be detected. The probe used for the integrated hybridization and detection system is immobilized on a substrate of a chip. The system includes a substrate 1, a probe 2 having a stem-loop structure, and a target molecule 3. Molecule G1 and molecule G2 are a pair of a fluorophore and a quencher and their relative position is interchangeable. Chemical group G4 is an exposed group on the substrate after a particular chemical treatment. Chemical group G3 is a chemical group attached to one end of the probe by chemical modification. Chemical group G3 and G4 can form strong covalent bond or non-covalent bond under specific conditions so that the probe can be immobilized on the substrate.

FIG. 2B illustrates a state after a probe, for use in an integrated hybridization and detection system, is hybridized to a target molecule. Because of the hybridization between the probe and the target molecule, the stem-loop structure shown in FIG. 2A is disrupted. Accordingly, the distance between the fluorophore and the quencher at the two ends of the probe becomes longer and the fluorescent emission from the fluorophore excited by a light source is no longer quenched by the quencher. The fluorescence emission can now be detected. The system includes a substrate 1, a probe 2 having a stem-loop structure, and a target molecule 3. Molecule G1 and molecule G2 are a pair of a fluorophore and a fluorescent quencher and their relative position is interchangeable.

Chemical group G4 is an exposed group on the substrate after a particular chemical treatment. Chemical group G3 is a chemical group attached to one end of the probe by chemical modification. Chemical group G3 and G4 can form strong covalent bond or non-covalent bond under specific conditions so that the probe can be immobilized on the substrate.

FIG. 3A illustrates a state before a pair of probes, which can be used for an integrated hybridization and detection system, are hybridized to a target molecule. The pair of probes includes a first probe comprising one end which can be covalently bond to a surface of a substrate modified by a particular chemical modification and the other end labeled with a first fluorophore; and a second probe in a liquid of the system having one end labeled with a second fluorophore. Hybridization of both the first probe and the second probe to a target molecule brings the two probes into close proximity to allow fluorescence resonance energy transfer between the two fluorophores to generate a detectable signal. The system includes substrate 1, probe 2 immobilized to the substrate, probe 3 in the liquid, and target molecule 4. Molecule G1 and G2 are a pair of fluorophores that allows fluorescence resonance energy transfer. Chemical group G4 is an exposed group on the substrate after a particular chemical treatment. Chemical group G3 is a chemical group attached to one end of the probe by chemical modification. Chemical group G3 and G4 can form strong covalent bond or non-covalent bond under specific conditions so that the probe can be immobilized on the substrate.

FIG. 3B illustrates a state after a pair of probes, which can be used for an integrated hybridization and detection system, are hybridized to a target molecule. The pair of the probes are in close proximity to each other after they are hybridized to the target molecule. The distance between the first and the second fluorophore are within the required distance allow fluorescence resonance energy transfer, i.e., within Förster radius. A fluorescent signal can be detected by applying a light source using the wavelength for exciting the first fluorophore and by receiving the signal using the emission wavelength of the second fluorophore. The system includes a substrate 1, a probe 2 immobilized to the substrate, a probe 3 in the liquid, and a target molecule 4. Molecule G1 and G2 are a pair of fluorophores that allows fluorescence resonance energy transfer. Chemical group G4 is an exposed group on the substrate after a particular chemical treatment. Chemical group G3 is a chemical group attached to one end of the probe by chemical modification. Chemical group G3 and G4 can form strong covalent bond or non-covalent bond under specific conditions so that the probe can be immobilized on the substrate.

FIG. 4A illustrates a state before a pair of probes, which can be used for an integrated hybridization and detection system, are hybridized to a target molecule. The pair of probes includes a first probe comprising one end which can be covalently bond to a surface of a substrate modified by a particular chemical modification and the other end labeled with a first fluorophore; and a second probe in a liquid phase of the system having one end labeled with a fluorescent quencher or a second fluorophore. When the target molecule is not in the system, the first probe hybridizes with the second probe and the fluorescence emission from the first fluorophore is quenched by the quencher or the excited energy of the first fluorophore is transferred to the second fluorophore via fluorescence resonance energy transfer, so that no emission signal from the first fluorophore is detected. The system includes: a substrate 1, a probe 2 immobilized on the substrate, a probe 3 which can be hybridized to the probe 2, and a target molecule 4. Molecule G1 and G2 are a pair of a fluorophore and a quencher or a pair of fluorophores that allows fluorescence resonance energy transfer. Chemical group G4 is an exposed group on the substrate after a particular chemical treatment. Chemical group G3 is a chemical group attached to one end of the probe by chemical modification. Chemical group G3 and G4 can form strong covalent bond or non-covalent bond under specific conditions so that the probe can be immobilized on the substrate.

FIG. 4B illustrates a state after a pair of probes, which can be used for an integrated hybridization and detection system, are hybridized to a target molecule. The pair of probes includes a first probe comprising one end which can be covalently bond to a surface of a substrate modified by a particular chemical modification and the other end labeled with a first fluorophore; and a second probe in a liquid of the system comprising one end labeled with a fluorescent quencher or a second fluorophore. When the target molecule is in the system, the hybridization between the first probe and the second probe is replaced by a hybridization between the first probe and the target molecule. The fluorescence emission from the first fluorophore is no longer quenched by the quencher or the excited energy of the first fluorophore is no longer transferred to the second fluorophore via fluorescence resonance energy transfer, so that the emission signal from the first fluorophore is detected. The system includes: a substrate 1, a probe 2 immobilized on the substrate, a probe 3 which can be hybridized to the probe 2, and a target molecule 4. Molecule G1 and G2 are a pair of a fluorophore and a quencher or a pair of fluorophores that allows fluorescence resonance energy transfer. Chemical group G4 is an exposed group on the substrate after a particular chemical treatment. Chemical group G3 is a chemical group attached to one end of the probe by chemical modification. Chemical group G3 and G4 can form strong covalent bond or non-covalent bond under specific conditions so that the probe can be immobilized on the substrate.

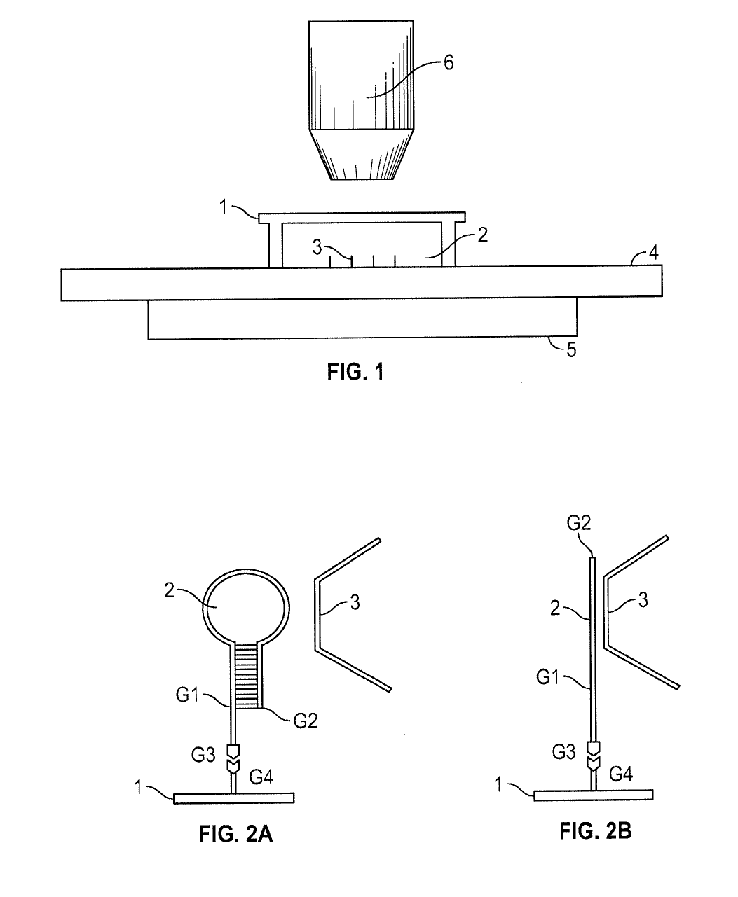

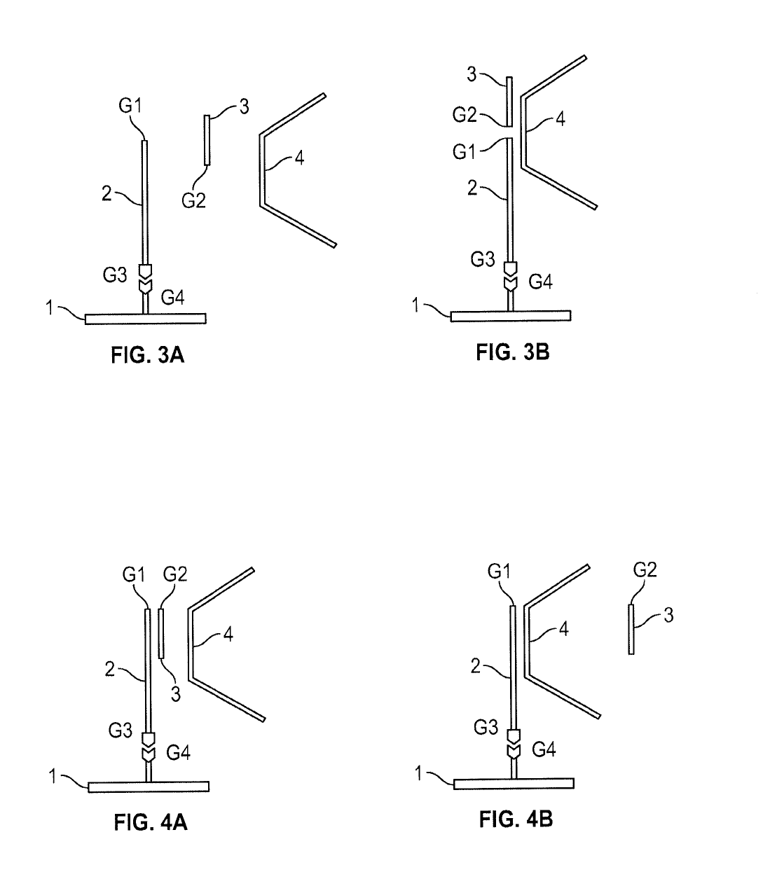

### MODES OF CARRYING OUT THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “a controllably closed space” means that the opening and closing of the space can be controlled at will, e.g., open to the outside to allow addition of sample or other reagents and close to allow the target nucleic acid preparation, amplification if desirable, and hybridization to a nucleic acid probe in the controllably closed space without any material exchange between the controllably closed space and the outside environment.

As used herein, “biocompatibility” refers to the quality and ability of a material of not having toxic or injurious effects on biological systems and biological or biochemical reactions.

As used herein, “thermal conductivity” refers to the effectiveness of a material as a thermal insulator, which can be expressed in terms of its thermal conductivity. The energy transfer rate through a body is proportional to the temperature gradient across the body and its cross sectional area. In the limit of infintesimal thickness and temperature difference, the fundamental law of heat conduction is:

Q=λAdT/dx

wherein Q is the heat flow, A is the cross-sectional area, dT/dx is the temperature/thickness gradient and λ is defined as the thermal conductivity value. A substance with a large thermal conductivity value is a good conductor of heat, one with a small thermal conductivity value is a poor heat conductor, i.e., a good insulator. Hence, knowledge of the thermal conductivity value (units W/m·K) allows comparisons, quantitative comparisons if desirable, to be made between the thermal insulation efficiencies of different materials.

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “probe” refers to an oligonucleotide or a nucleic acid that hybridizes to a target sequence, typically to facilitate its detection. The term “target sequence” refers to a nucleic acid sequence to which the probe specifically binds. Unlike a primer that is used to prime the target nucleic acid in amplification process, a probe need not be extended to amplify target sequence using a polymerase enzyme.

As used herein, “complementary or matched” means that two nucleic acid sequences have at least 50% sequence identity. Preferably, the two nucleic acid sequences have at least 60%, 70,%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein: “stringency of hybridization” in determining percentage mismatch is as follows:

1) high stringency: 0.1×SSPE (or 0.1×SSC), 0.1% SDS, 65° C.;

2) medium stringency: 0.2×SSPE (or 1.0×SSC), 0.1% SDS, 50° C. (also referred to as moderate stringency); and

3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “gene chip” refers to an array of oligonucleotides or nucleic acids, e.g., long-chain PCR products, immobilized on a surface that can be used for any suitable purpose. Exemplary uses of a gene chip include screening an RNA sample (after reverse transcription) and thus a method for rapidly determining which genes are being expressed in the cell or tissue from which the RNA came, single nucleotide polymorphism (SNP), detection, mutation analysis, disease or infection prognosis or diagnosis, genome comparisons, etc.

As used herein, “melting temperature” (“Tm”) refers to the midpoint of the temperature range over which nucleic acid duplex, i.e., DNA:DNA, DNA:RNA, RNA:RNA, PNA: DNA, LNA:RNA and LNA: DNA, etc., is denatured.

As used herein, “label” refers to any chemical group or moiety having a detectable physical property or any compound capable of causing a chemical group or moiety to exhibit a detectable physical property, such as an enzyme that catalyzes conversion of a substrate into a detectable product. The term “label” also encompasses compound that inhibit the expression of a particular physical property. The “label” may also be a compound that is a member of a binding pair, the other member of which bears a detectable physical property. Exemplary labels include mass groups, metals, fluorescent groups, luminescent groups, chemiluminescent groups, optical groups, charge groups, polar groups, colors, haptens, protein binding ligands, nucleotide sequences, radioactive groups, enzymes, particulate particles, a fluorescence resonance energy transfer (FRET) label, a molecular beacon and a combination thereof.

As used herein, “microarray chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, can be incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips, upon which the processes can be carried out, can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces.

As used herein, “microlocations” refers to places that are within, on the surface or attached to the substrate wherein the microarray chips and/or other structures or devices are located. As used herein, “distinct microlocations” means that the microlocations are sufficiently separated so that, if needed, reagents can be added and/or withdrawn and reactions can be conducted in one microlocation independently from another microlocation. It is not necessary that each microlocation is “distinct” from all other microlocations, although in certain embodiments, each microlocation can be “distinct” from all other microlocations.

As used herein, “microlocations are in a well format” means that there are indentations with suitable three dimensional shape at the microlocations so that microarray chips and/or other structures or devices such as temperature controllers, can be built or placed into.

As used herein, “microlocations is thermally insulated” means that the microlocations have certain structures or substances that can be used to adjust to and maintain temperature at a microlocation at a desired level independently from other microlocations or any place outside the microlocation.

As used herein, “sample” refers to anything which may contain a target nucleic acid and protein or extracted nucleic acid and protein to be analyzed using the present lab-on-chip systems and/or methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “assessing” refers to quantitative and/or qualitative determination of the hybrid formed between the probe and the target nucleotide sequence, e.g., obtaining an absolute value for the amount or concentration of the hybrid, and also of obtaining an index, ratio, percentage, visual or other value indicative of the level of the hybrid. Assessment may be direct or indirect and the chemical species actually detected need not of course be the hybrid itself but may, for example, be a derivative thereof, reduction or disappearance of the probe and/or the target nucleotide sequence, or some further substance.

### B. LAB-ON-CHIP SYSTEMS AND METHODS FOR ANALYZING A NUCLEIC ACID

In one aspect, the present invention is directed to a lab-on-chip system for analyzing a nucleic acid, which system comprises a controllably closed space enclosed by a suitable material on a substrate, wherein said suitable material is thermoconductive, biocompatible and does not inhibit nucleic acid amplification or hybridization, and said controllably closed space comprising, on the surface of said substrate, a nucleic acid probe complementary to a target nucleic acid and, on or off the surface of said substrate, other reagents suitable for preparation of said target nucleic acid from a sample, amplification of said target nucleic acid, hybridization between said nucleic acid probe and said target nucleic acid, and/or means for detecting hybridization between said nucleic acid probe and said target nucleic acid, and wherein addition of a sample comprising said target nucleic acid into said controllably closed space, under suitable conditions, results in continuous sample preparation from said sample and/or amplification of said prepared target nucleic acid, and hybridization between said nucleic acid probe and said target nucleic acid, and preferably the detection of the hybridization signal, in said controllably closed space without any material exchange between said controllably closed space and the outside environment.

Any suitable material can be used in the present lab-on-chip systems. Preferably, the suitable material is an air-tight material, e.g., MJ Research self seal chamber and self seal gel or an enclosed plastic lumen and the like. Also preferably, a waterproof material is used in the present lab-on-chip systems.

The suitable material can be connected to the substrate to form the controllably closed space by any suitable methods. For example, the suitable material can be glued on the substrate to form the controllably closed space. In another example, the suitable material can be microfabricated on the substrate to form the controllably closed space.

Any suitable substrate can be used in the present lab-on-chip systems. For example, the substrate can comprise a material selected from the group consisting of a silicon, a plastic, a glass, a quartz glass, a ceramic, a rubber, a metal, a polymer and a combination thereof.

The present lab-on-chip systems can comprise a single nucleic acid probe on the substrate. Alternatively, the present lab-on-chip systems can comprise a plurality of nucleic acid probes on the substrate to analyze a plurality of target nucleic acids, preferably simultaneously.

Both single-stranded or double-stranded probes can be used in the present lab-on-chip systems. The probes can be oligonucleotides or other types of nucleic acids, e.g., long-chain PCR products. The nucleic acid probes used in the present lab-on-chip systems can have any suitable length. When a single-stranded probe is used, it preferably has a length ranging from about 5 nt to about 100 nt. When a double-stranded probe is used, it preferably has a length ranging from about 50 basepairs to about 3,000 basepairs. The nucleic acid probes used in the present lab-on-chip systems can be labeled. Any suitable labels can be used. Exemplary labels include a radioactive label, a fluorescent label, a chemical label, an enzymatic label, a luminescent label, a fluorescence resonance energy transfer (FRET) label and a molecular beacon.

The nucleic acid probe can be attached to the substrate via any suitable means. For example, the nucleic acid probe can be modified to facilitate its attachment to the substrate. In another example, the nucleic acid probe can be attached to the substrate via a functional group on the substrate, e.g., —CHO, —NH₂, —SH or —S—S— group. In still another example, the nucleic acid probe can be attached to the substrate via a binding pair, e.g., biotin/avidin pair or biotin/streptavidin pair. In yet another example, the nucleic acid probe can be attached to the substrate via ultraviolet-activated crosslinking, heat-activated crosslinking, an interaction between NH₂ and —CHO, an interaction between —SH and —SH, an interaction between biotin and avidin and an interaction between biotin and streptavidin.

The nucleic acid probe can be a specific or degenerate probe. The nucleic acid probe can be DNA, RNA or a combination thereof. The nucleic acid probe can be substantially complementary to or perfectly match the target nucleic acid.

In a specific embodiment, the present lab-on-chip system, for a detecting position, can comprise two nucleic acid probes, wherein a first probe comprises a first FRET label and is attached to the substrate and a second probe comprises a second FRET label in liquid, and hybridization of both the first and the second probes to a target nucleic acid brings the two probes into close proximity to allow fluorescence resonance energy transfer between the two probes to generate a detectable signal. Any suitable FRET labels can be used. Preferably, a combination of Fluorescein and TAMRA, TAMRA and Cy5, ROX and Cy5, IAEDANS and Fluorescein, or Fluorescein and QSY-7, is used.

In another specific embodiment, the present lab-on-chip system, for a detecting position, can comprise two nucleic acid probes, wherein the first probe is attached to the substrate and the second probe is in a liquid, the two probes are complementary to each other and the first probe is complementary to a target nucleic acid, the Tm of a hybrid of the two probes is about 5° C. to about 30° C. lower than that of a hybrid of the target nucleic acid and the first probe, the first probe comprises a fluorescent label and the second probe comprises a quencher for the fluorescent label, and wherein in the absence of the target nucleic acid, the two probes are hybridized and the fluorescent label is quenched by the quencher, and in the presence of a target nucleic acid, the probes are separated by the hybridization of the first probe to the target nucleic acid, and the fluorescent label is no longer quenched by the quencher to generate a detectable signal. Any suitable fluorescent label, e.g., 6-FAM, TET, HEX, Cy3, Cy5, Texas Red, ROX, Fluorescein or TAMRA, and any suitable quencher for the fluorescent label e.g., Dabcyl, Black Hole-1, Black Hole-2 or a gold particle with a diameter from about 0.1 nm to about 10 nm, can be used.

If desirable, the target nucleic acid can be amplified by any suitable methods, e.g., polymerase chain reaction (PCR), ligase chain reaction (LCR), nucleic acid sequence-based amplification (NASBA), strand displacement amplification (SDA), transcription-medicated amplification (TMA) and rolling cycle amplification (RCA). To effect the amplification, the present lab-on-chip systems can comprise a buffer, as well as any other reagents, suitable for at least one of the target nucleic acid amplification methods.

In a specific embodiment, the present lab-on-chip systems can comprise reagents suitable for amplification of the target nucleic acid and hybridization between a nucleic acid probe and the target nucleic acid. In another specific embodiment, the present lab-on-chip systems can comprise reagents suitable for preparation of the target nucleic acid from a sample, amplification of the target nucleic acid, and hybridization between said nucleic acid probe and the target nucleic acid.

The present lab-on-chip systems can further comprise a temperature controlling device, e.g., a temperature controlling device comprising a temperature controlling unit of a commercially available PCR machine or a water bath. The present lab-on-chip systems can further comprise a signal detecting device, e.g., a fluorescent imaging device.

The present lab-on-chip systems can be used for any suitable purpose(s). For example, the present lab-on-chip systems can be used for continuous sample preparation of the target nucleic acid from the sample and hybridization between the nucleic acid probe and the prepared target nucleic acid in the controllably closed space. In another example, the present lab-on-chip systems can be used for continuous hybridization between the nucleic acid probe and a prepared target nucleic acid and hybridization signal analysis in the controllably closed space.

The present lab-on-chip systems can further comprise an instruction for preparing, amplifying and/or hybridizing a target nucleic acid in a sample using the system. In another aspect, the present invention is directed to a method for analyzing a nucleic acid, which method comprises: a) providing an above-described lab-on-chip system; b) adding a sample containing or suspected of containing a target nucleic acid into said controllably closed space of said system provided in a); and c) allowing continuous sample preparation of said target nucleic acid from said sample and/or amplification of said prepared target nucleic acid, and hybridization between said nucleic acid probe and said prepared target nucleic acid, and preferably the detection of the hybridization signal, in said controllably closed space.

Preferably, the present method can further comprise amplifying the target nucleic acid in the controllably closed space. Also preferably, the present method can further comprise analyzing hybridization between the nucleic acid probe and the prepared target nucleic acid in the controllably closed space.

Target nucleotide sequences that can be analyzed and/or quantified using the present lab-on-chip systems and methods can be DNA, RNA or any other naturally or synthetic nucleic acid sample. Test samples can include body fluids, such as urine, blood, semen, cerebrospinal fluid, pus, amniotic fluid, tears, or semisolid or fluid discharge, e.g., sputum, saliva, lung aspirate, vaginal or urethral discharge, stool or solid tissue samples, such as a biopsy or chorionic villi specimens. Test samples also include samples collected with swabs from the skin, genitalia, or throat. Test samples can be processed to isolate nucleic acid by a variety of means well known in the art.

Similarly, although the present lab-on-chip systems and methods can be used to analyze a single sample with a single probe at a time. Preferably, the present method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously. Any suitable target nucleic acids can be analyzed using the present lab-on-chip systems and methods. Exemplary target nucleic acids include DNA, such as A-, B- or Z-form DNA, and RNA such as mRNA, tRNA and rRNA. The nucleic acids can be single-, double- and triple-stranded nucleic acids. In addition, target nucleic acids encoding proteins and/or peptides can be analyzed. Exemplary proteins or peptides include enzymes, transport proteins such as ion channels and pumps, nutrient or storage proteins, contractile or motile proteins such as actins and myosins, structural proteins, defense proteins or regulatory proteins such as antibodies, hormones and growth factors.

Any suitable samples can be analyzed using the present lab-on-chip systems and methods. Preferably, a biosample is analyzed using the present lab-on-chip systems and methods. For example, a biosample of plant, animal, human, fungus, bacterium and virus origin can analyzed. If a sample of a mammal or human origin is analyzed, the sample can be derived from a particular tissue or organ. Exemplary tissues include connective, epithelium, muscle or nerve tissue. Exemplary organs include eye, annulospiral organ, auditory organ, Chievitz organ, circumventricular organ, Corti organ, critical organ, enamel organ, end organ, external female gential organ, external male genital organ, floating organ, flower-spray organ of Ruffini, genital organ, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmüller, sense organ, organ of smell, spiral organ, subcommissural organ, subfornical organ, supernumerary organ, tactile organ, target organ, organ of taste, organ of touch, urinary organ, vascular organ of lamina terminalis, vestibular organ, vestibulocochlear organ, vestigial organ, organ of vision, visual organ, vomeronasal organ, wandering organ, Weber organ and organ of Zuckerkandl. Preferably, samples derived from an internal mammalian organ such as brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc, are analyzed.

Alternatively, pathological samples in connection with various diseases or disorders or infections can be analyzed. Exemplary diseases or disorders include neoplasms (neoplasia), cancers, immune system diseases or disorders, metabolism diseases or disorder, muscle and bone diseases or disorders, nervous system diseases or disorders, signal diseases or disorders and transporter diseases or disorders. The infection to be analyzed can be fungal, bacterial and viral infection.

### C. EXAMPLES

To address the drawbacks of the existing lab-on-chip systems, we developed an exemplary lab-on-chip system which integrates conventional three-step nucleic acid analysis (sample preparation, nucleic acid hybridization, and hybridization signal detection) in one controllably closed space without any material exchange between the controllably closed space and the outside environment. The system reduces or avoids introduction of experimental error and contamination. After the analysis, the chip in the system can be discarded. Because the system is closed, there is no residual contamination which is often seen in conventional nucleic acid analysis. The whole process can be finished within three hours or less.

### Example 1: A Lab-on-chip System Based on Fluorescence Resonance Energy Transfer (FRET) for use in Detection of Hepatitis B Virus

1. Preparation of a substrate having an aldehyde group

A glass substrate was soaked in an acidic wash solution at room temperature overnight. The glass substrate was then rinsed with water, washed three times with distilled water, and washed two times with deionized water. It was then dried by centrifugation followed by heating to 110° C. for 15 minutes. The glass substrate was soaked in 1% APTES in 95% ethanol and was shaken gently in a shaker for one hour at room temperature. After soaking in 95% ethanol, the glass substrate was rinsed and then dried in a vacuum drier at −0.08 Mpa to −0.1 Mpa and 110° C. for twenty minutes. Once the glass substrate was cooled to room temperature, it was soaked in 12.5% glutaraldehyde solution (for 400 ml 12.5% glutaraldehyde solution, mix 100 ml 50% glutaraldehyde with 300 ml sodium phosphate buffer (1M NaH₂PO₄ 30 ml and 2.628 g NaCl, adjust pH to 7.0)). After soaking for 4 hours at room temperature, the solution was shaken gently and the glass substrate was taken out of the glutaraldehyde solution and washed once in 3×SSC, followed by two washes in deionized water. The excess water was removed by centrifugation and the glass plate was dried at room temperature.

2. Synthesis of primers and probes

The primers and the probes were synthesized by Shanghai BioAsia Biotechnology Co. Probe 1 is amino-5′-polyT(15nt)

GCATGGACATCGACCCTTATAAAG-3′-TAMRA (SEQ ID NO:1). Probe 2 is Cy5-5′-GGAGCTACTGTGGAGTTACTC CTGG-3′ (SEQ ID NO:2). The upstream primer is gTTCAAgCCTCCAAgCTgTg (SEQ ID NO:3). The down stream primer is TCAgAAggCAAAAAAgAgAgTAACT (SEQ ID NO:4).

3. Preparation of the glass substrate having probes immobilized on the surface Probe 1 is dissolved in 50% DMSO with final concentration at 10 μM. The probes were printed on the substrate using a microarray printing device (Cartesian Technologies, Calif., U.S.A.) according to a pre-designed pattern. The printed substrate was then dried overnight at room temperature. The printed substrate was then soaked twice in 0.2% SDS at room temperature for 2 minutes with shaking. The substrate was rinsed twice and washed once with deionized water and then dried by centrifugation. The substrate was then transferred to a NaBH₄ solution (0.1 g NaBH₄ dissolved in 300 ml 1×PBS and 100 ml ethanol) and shaken gently at room temperature for 5 minutes. The substrate was again rinsed twice and washed twice with deionized water for 1 minute of each wash and dried by centrifugation.

4. Preparation of reaction chamber

The reaction chamber was prepared using self seal chamber (MJ Research, Inc., Mass., U.S.A.) according to the operation manual. The substrate having the immobilized probes was made to face the inside of the chamber.

5. Nucleic acid amplification and hybridization

PCR reaction system included: 10 mmol/L Tris-HC1 (pH 8.3 at 24° C.), 50 mmol/L KC1, 1.5 mmol/L MgCl₂, 0.5 μmol/L of upstream primer and downstream primer, 1 unit Taq DNA polymerase, 200 μmol/L dNTPs (dATP, dTTP, dCTP, and dGTP), 0.1% BSA, 0.1% Tween 20, 2 μmol/L probe 2. The total reaction volume is 25 μl. The PCR reaction system was then introduced into the reaction chamber and sealed. The PCR was carried using PTC-200 (MJ Research Inc.) with a program: predenaturing at 94° C. for 1 minute; main cycle at 94° C. for 30 sec, 55° C. for 30 sec, and 72° C. for 1 minute for 30 cycles; and at 72° C. for 10 minutes. After the PCR reaction, hybridization was preformed using the same PCR machine at 52° C. for 4 hours.

6. Hybridization signal detection

The hybridization signal was detected using ScanArray 4000 fluorescence scanner (GSI Lumonics, Mass., USA). Laser device 3 was chosen with an exciting wavelength at 543 nm. Optical filter 7 was used for signal detection. The function of the laser device and the light-electric multiplier tube was chosen at 80%. The focal setting was adjusted according to the glass substrate. Detection process was performed according to the operation manual. A chip from the reaction chamber having a sample added to the reaction system showing a relatively strong fluorescence signal at the position of the probe on the substrate and a relatively weak fluorescence signal at the position of a negative control probe, while a chip from the reaction chamber without the sample showing a relatively weak fluorescence signal, indicates that the sample contains nucleic acids of hepatitis B virus.

### Example 2: A lab-on-chip System Based on Molecular Beacon for Use in Detection of Vepatitis B Virus

1. Preparation of a substrate having an aldehyde group

A glass substrate was soaked in an acidic wash solution at room temperature overnight. The glass substrate was then rinsed with water, washed three times with distilled water, and washed two times with deionized water. It was then dried by centrifugation followed by heating to 110° C. for 15 minutes. The glass substrate was soaked in 1% APTES in 95% ethanol and was shaken gently in a shaker for one hour at room temperature. After soaking in 95% ethanol, the glass substrate was rinsed and then dried in vacuum drier at −0.08 Mpa to −0.1 Mpa and 110° C. for twenty minutes. Once the glass substrate was cooled to room temperature, it was soaked in 12.5% glutaraldehyde solution (for 400 ml 12.5% glutaraldehyde solution, mix 100 ml 50% glutaraldehyde with 300 ml sodium phosphate buffer (1M NaH₂PO₄ 30 ml and 2.628 g NaCl, adjust pH to 7.0)). After soaking for 4 hours at room temperature, the solution was shaken gently and the glass substrate was taken out of the glutaraldehyde solution and washed once in 3×SSC, followed by twice in deionized water. The excess water was removed by centrifugation and the glass plate was dried at room temperature.

2. Synthesis of primers and probes

The primers and the probes were synthesized by Shanghai BioAsia Biotechnology Co. The molecular beacon is 5′-amino-TTTTT TTTTT TTTT [symbol] CGTGC-GTTCAAgCCTCCAAgCTgTg-GCACG A-3′-TAMRA (SEQ ID NO:5). Nucleotide [symbol] is labeled with a fluorescence quencher Dabcyl. The upstream primer is gTTCAAgCCTCCAAgCTgTg (SEQ ID NO:6). The down stream primer is TCAgAAggCAAAAAAgAgAgTAACT (SEQ ID NO:7).

3. Preparation of the glass substrate having probes immobilized on the surface

The molecular beacon probe is dissolved in 50% DMSO with final concentration at 10 μM. The probes were printed on the substrate using microarray printing device (Cartesian Technologies, Calif., U.S.A.) according to a pre-designed pattern. The printed substrate was then dried overnight at room temperature. The printed substrate was then soaked twice in 0.2% SDS at room temperature for 2 minutes with shaking. The substrate was rinsed twice and washed once with deionized water and then dried by centrifugation. The substrate was then transferred to a NaBH₄ solution (0.1 g NaBH₄ dissolved in 300 ml 1×PBS and 100 ml ethanol) and shaken gently at room temperature for 5 minutes. The substrate was again rinsed twice and washed twice with deionized water for 1 minute of each wash and dried by centrifugation.

4. Preparation of reaction chamber

The reaction chamber was prepared using self seal chamber (MJ Research, Inc., Mass., U.S.A.) according to the operation manual. The substrate having the immobilized probes was made to face the inside of the chamber.

5. Nucleic acid amplification and hybridization

PCR reaction system included: 10 mmol/L Tris-HCl (pH 8.3 at 24° C.), 50 mmol/L KCl, 1.5 mmol/L MgCl₂, 0.5 μmol/L of upstream primer and downstream primer, 1 unit Taq DNA polymerase, 200 μmol/L dNTPs (dATP, dTTP, dCTP, and dGTP), 0.1% BSA, 0.1% Tween 20. The total reaction volume is 25 μl. The PCR reaction system was then introduced into the reaction chamber and sealed. The PCR was carried using PTC-200 (MJ Research Inc.) with a program: predenaturing at 94° C. for 1 minute; main cycle at 94° C. for 30 sec, 55° C. for 30 sec, and 72° C. for 1 minute for 30 cycles; and at 72° C. for 10 minutes. After the PCR reaction, hybridization was preformed using the same PCR machine at 52° C. for 4 hours.

6. Hybridization signal detection

The hybridization signal was detected using ScanArray 4000 (GSI Lumonics, MA, USA). Laser device 3 was chosen with an exciting wavelength at 543 nm. Optical filter 7 was used for signal detection. The function of the laser device and the light-electric multiplier tube was chosen at 80%. The focal setting was adjusted according to the glass substrate. Detection process was performed according to the operation manual. A chip from the reaction chamber having a sample added to the reaction system showing a relatively strong fluorescence signal at the position of the probe on the substrate and a relatively weak fluorescence signal at the position of a negative control probe, while a chip from the reaction chamber without the sample showing a relatively weak fluorescence signal, indicates that the sample contains nucleic acids of hepatitis B virus.

### Example 3: A Fluorescence Quenching Based Lab-on-chip System for Detection of Hepatitis B Virus

1. Preparation of a substrate having an aldehyde group

A glass substrate was soaked in an acidic wash solution at room temperature overnight. The glass substrate was then rinsed with water, washed three times with distilled water, and washed two times with deionized water. It was then dried by centrifugation followed by heating to 110° C. for 15 minutes. The glass substrate was soaked in 1% APTES in 95% ethanol and was shaken gently in a shaker for one hour at room temperature. After soaking in 95% ethanol, the glass substrate was rinsed and then dried in a vacuum drier at −0.08 Mpa to −0.1 Mpa and 110° C. for twenty minutes. Once the glass substrate was cooled to room temperature, it was soaked in 12.5% glutaraldehyde solution (for 400 ml 12.5% glutaraldehyde solution, mix 100 ml 50% glutaraldehyde with 300 ml sodium phosphate buffer (1M NaH₂PO₄ 30 ml and 2.628 g NaCl, adjust pH to 7.0)). After soaking for 4 hours at room temperature, the solution was shaken gently and the glass substrate was taken out of the glutaraldehyde solution and washed once in 3×SSC, followed by twice in deionized water. The excess water was removed by centrifugation and the glass plate was dried at room temperature.

2. Synthesis of primers and probes

The primers and the probes were synthesized by Shanghai BioAsia Biotechnology Co. Probe 1 is amino-5′-polyT(15 nt) GCATGGACATCGACCCTTATAAAG -3′-TAMRA (SEQ ID NO:8). Probe 3 is 5′- [symbol] CTTTATAAGGGTCG cct-3′ (SEQ ID NO:9) Nucleotide [symbol] is labeled with a fluorescence quencher Dabcyl. The upstream primer is gTTCAAgCCTCCAAgCTgTg (SEQ ID NO:10). The down stream primer is TCAgAAggCAAAAAAgAgAgTAACT (SEQ ID NO:11).

3. Preparation of the glass substrate having probes immobilized on the surface

Probe 1 is dissolved in 50% DMSO with final concentration at 10 μM. The probes were printed on the substrate using a microarray printing device (Cartesian Technologies, Calif., U.S.A.) according to a pre-designed pattern. The printed substrate was then dried overnight at room temperature. The printed substrate was then soaked twice in 0.2% SDS at room temperature for 2 minutes with shaking. The substrate was rinsed twice and washed once with deionized water and then dried by centrifugation. The substrate was then transferred to a NaBH₄ solution (0.1 g NaBH₄ dissolved in 300 ml 1×PBS and 100 ml ethanol) and shaken gently at room temperature for 5 minutes. The substrate was again rinsed twice and washed twice with deionized water for 1 minute of each wash and dried by centrifugation.

4. Preparation of reaction chamber

The reaction chamber was prepared using self seal chamber (MJ Research, Inc., Mass., U.S.A.) according to the operation manual. The substrate having the immobilized probes was made to face the inside of the chamber.

5. Nucleic acid amplification and hybridization

PCR reaction system included: 10 mmol/L Tris-HCl (pH 8.3 at 24° C.), 50 mmol/L KCl, 1.5 mmol/L MgCl₂, 0.5 μmol/L of upstream primer and downstream primer, 1 unit Taq DNA polymerase, 200 μmol/L dNTPs (dATP, dTTP, dCTP, and dGTP), 0.1% BSA, 0.1% Tween 20, 2 μmol/L probe 3. The total reaction volume is 25 μl. The PCR reaction system was then introduced into the reaction chamber and sealed. The PCR was carried using PTC-200 (MJ Research Inc.) with a program: predenaturing at 94° C. for 1 minute; main cycle at 94° C. for 30 sec, 55° C. for 30 sec, and 72° C. for 1 minute for 30 cycles; and at 72° C. for 10 minutes. After the PCR reaction, hybridization was preformed using the same PCR machine at 52° C. for 4 hours. Then the reaction was incubated at 30° C. for 5 minutes to allow binding of probe 3 to hybridized probe 1.

6. Hybridization signal detection

The hybridization signal was detected using ScanArray 4000 (GSI Lumonics, Mass., USA). Laser device 3 was chosen with an exciting wavelength at 543 nm. Optical filter 7 was used for signal detection. The function of the laser device and the light-electric multiplier tube was chosen at 80%. The focal setting was adjusted according to the glass substrate. Detection process was performed according to the operation manual. A chip from the reaction chamber having a sample added to the reaction system showing a relatively strong fluorescence signal at the position of the probe on the substrate and a relatively weak fluorescence signal at the position of a negative control probe, while a chip from the reaction chamber without the sample showing a relatively weak fluorescence signal, indicates that the sample contains nucleic acids of hepatitis B virus.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

What is claimed is:

1. A method for analyzing a nucleic acid, which method comprises:
a) providing a lab-on-chip system for analyzing a nucleic acid, which system comprises a controllably closed space enclosed by a suitable material on a substrate, wherein said suitable material is thermoconductive, biocompatible and does not inhibit nucleic acid amplification or hybridization, and said controllably closed space comprising, on the surface of said substrate, a nucleic acid probe complementary to a target nucleic acid and, on or off the surface of said substrate, other reagents suitable for preparation of said target nucleic acid from a sample, amplification of said target nucleic acid, hybridization between said nucleic acid probe and said target nucleic acid, and/or means for detecting hybridization between said nucleic acid probe and said target nucleic acid, and wherein addition of a sample comprising said target nucleic acid into said controllably closed space, under suitable conditions, results in continuous sample preparation from said sample and/or amplification of said prepared target nucleic acid, and hybridization between said nucleic acid probe and said target nucleic acid, and preferably detection of the hybridization signal, in said controllably closed space without any material exchange between said controllably closed space and the outside environment;
b) adding a sample containing or suspected of containing a target nucleic acid into said controllably closed space of said system provided in a); and
c) allowing continuous sample preparation of said target nucleic acid from said sample and/or amplification of said prepared target nucleic acid, and hybridization between said nucleic acid probe and said prepared target nucleic acid, and preferably detection of the hybridization signal, in said controllably closed space.
